(19) United States
(12) Patent Application Publication
(10) Pub. No.: US 2025/0000218 A1
(43) Pub. Date: Jan. 2, 2025

(54) WEARABLE RING DEVICE WITH DEFORMABLE INNER COVER

(71) Applicant: Oura Health Oy, Oulu (FI)

(72) Inventors: Antti Kalevi Lämsä, Oulu (FI); Sami Ihme, Oulu (FI)

(21) Appl. No.: 18/344,532

(22) Filed: Jun. 29, 2023

(51) Int. Cl. A44C 9/00; A44C 9/02

(52) U.S. Cl. CPC A44C 9/0053; A44C 9/02; A61B 5/256

(57) ABSTRACT

Systems and devices for a wearable ring device are described. The wearable ring device may include a housing comprising one or more sensors configured to acquire physiological data from a user and a deformable material extending along one or more portions of an inner circumferential surface of the housing. The deformable material may be configured to exert pressure on a digit of the user by conforming to an outer circumferential surface of the digit to form an interference fit with the digit of the user when the wearable ring device is worn by the user. In some cases, the deformable material may be arranged to enhance a signal quality of measurements by the one or more sensors through an application of pressure on the digit of the user within an area of the digit adjacent to the one or more sensors.

### FIELD OF TECHNOLOGY

The following relates to wearable devices and data processing, including a wearable ring device with deformable inner cover.

### BACKGROUND

Some wearable devices may be configured to collect physiological data from users, including heart rate, motion data, temperature data, photoplethysmogram (PPG) data, and the like. In some cases, some wearable devices may fit too loose or too tight, thereby affecting the quality of the physiological data collected. As such, techniques for improving the fit of the wearable device may be desired.

### BRIEF DESCRIPTION OF THE DRAWINGS

FIG. 1 illustrates an example of a system that supports a wearable ring device with deformable inner cover in accordance with aspects of the present disclosure.

FIG. 2 illustrates an example of a system that supports a wearable ring device with deformable inner cover in accordance with aspects of the present disclosure.

FIG. 3A shows an example of a side view of a wearable ring device that supports a wearable ring device with deformable inner cover in accordance with aspects of the present disclosure.

FIG. 3B shows an example of a partial top down view of a wearable ring device that supports a wearable ring device with deformable inner cover in accordance with aspects of the present disclosure.

FIG. 4A shows an example of a side view of a wearable ring device that supports a wearable ring device with deformable inner cover in accordance with aspects of the present disclosure.

FIG. 4B shows an example of a partial top down view of a wearable ring device that supports a wearable ring device with deformable inner cover in accordance with aspects of the present disclosure.

FIG. 5A shows an example of a side view of a wearable ring device that supports a wearable ring device with deformable inner cover in accordance with aspects of the present disclosure.

FIG. 5B shows an example of a partial top down view of a wearable ring device that supports a wearable ring device with deformable inner cover in accordance with aspects of the present disclosure.

FIG. 6A shows an example of a side view of a wearable ring device that supports a wearable ring device with deformable inner cover in accordance with aspects of the present disclosure.

FIG. 6B shows an example of a partial top down view of a wearable ring device that supports a wearable ring device with deformable inner cover in accordance with aspects of the present disclosure.

FIG. 7A shows an example of a side view of a wearable ring device that supports a wearable ring device with deformable inner cover in accordance with aspects of the present disclosure.

FIG. 7B shows an example of a partial top down view of a wearable ring device that supports a wearable ring device with deformable inner cover in accordance with aspects of the present disclosure.

FIG. 8A shows an example of a side view of a wearable ring device that supports a wearable ring device with deformable inner cover in accordance with aspects of the present disclosure.

FIG. 8B shows an example of a partial top down view of a wearable ring device that supports a wearable ring device with deformable inner cover in accordance with aspects of the present disclosure.

### DETAILED DESCRIPTION

In some wearable ring devices (e.g., wearables having sensors for measuring physiological parameters from a user), surfaces of the wearable ring device, including an inner surface of the wearable ring device, may generally be a non-deformable, hard surface that contacts the finger of the user. For example, in some versions of a wearable ring device, the inner surface of the wearable ring device is made of a hard material (e.g., an epoxy or metallic material) that maintains the same diameter regardless of a change in size of the finger due to environmental factors, differences in the shape of each user's finger, and the like.

As such, the wearable ring device fit may become loose when the user's finger shrinks, or the ring may become too tight if the finger swells. For example, the inside surface of the wearable ring device may not contact the finger when the user's finger shrinks due to a change (e.g., decrease) in temperature, a change in hydration levels, a decrease in salt intake, weight loss, and the like. In other cases, the ring may become too tight around the user's finger as the size of the finger swells due to an increase in temperature caused by exercise, warm weather, stress, and the like. Previous versions of the wearable ring device may include standard sizing and/or fit due to the hard inside material of the wearable ring device that does not change the fit of the wearable ring device over time or based on the size/shape of a user's finger.

Such a limitation may result in an inaccurate fit of the wearable ring device which may affect the ability of the wearable ring device to efficiently and accurately acquire physiological data. Moreover, wearing a wearable ring device that isn't the right size (e.g., too tight around the user's finger or too loose around the user's finger) may result in inaccurate physiological data readings, which may lead to a distorted picture of the user's overall health, as well as increased power consumption and decreased battery life.

Accordingly, to facilitate improved physiological measurements, aspects of the present disclosure are directed to improving the fit of the wearable ring device by using a material on the inner surface of the wearable ring device that allows the finger to expand or contract in size without affecting the fit of the wearable ring device. The material may be an example of a memory shape polymer that changes shape based on the body temperature of the user. For example, if the user's finger swells due to an increase in body temperature, then the increase in skin temperature may heat the memory shape polymer to adjust the size (e.g., contract the material on the inner surface) of the wearable ring device and accommodate the swelling. In other examples, if the user's finger shrinks due to a decrease in body temperature, then the ambient temperature of an air gap present between the user's finger and the wearable ring device may cool the memory shape polymer to adjust the size (e.g., expand the material on the inner surface) of the wearable ring device and accommodate the finger shrinking. In such cases, the material has the ability to return from a deformed state to the original shape when induced by an external stimulus such as temperature change. For example, the material may adjust the fit of the wearable ring device as the user moves between different environments, thereby increasing the adaptability and versatility of the wearable ring device.

In other examples, the material may be an example of a soft polymer material that may respond to mechanical stress and allow a comfortable fit even though the inner diameter of the wearable ring device changes. The material may extend around the entire circumference of the inner surface or over certain portions of the inner surface (e.g., over the sensor domes, on half of the circumference, or as dispersed pads on top of the internal surface).

By using a deformable material on the inner surface of the wearable ring device, the fit of the wearable ring device may be improved even when the finger diameter changes due to weather changes, other environmental factors, physiological changes associated with the user, and the like. In particular, manufacturing the inner circle of the wearable ring device with a material that allows the finger to change shape while the deformable material adapts to the anatomy of the finger may lead to more accurate physiological data measurements based on the personalized, improved fit, thereby increasing the efficiency and accuracy of the signal. The material on the inner surface of the wearable ring device may be positioned or otherwise configured to not interfere with the measurements by the sensors. For example, if the area around a light emitting component or light receiving component is made from a deformable material, the thickness or other geometric properties of the deformable material may be configured so that even when deformed, the material does not interfere with light emission or reception at the sensor components, thereby not interfering with the physiological measurements.

Aspects of the disclosure are initially described in the context of systems supporting physiological data collection from users via wearable devices. Aspects are then described in the context of wearable ring devices. Aspects of the disclosure are further illustrated by and described with reference to apparatus diagrams, system diagrams, and flowcharts that relate to a wearable ring device with deformable inner cover.

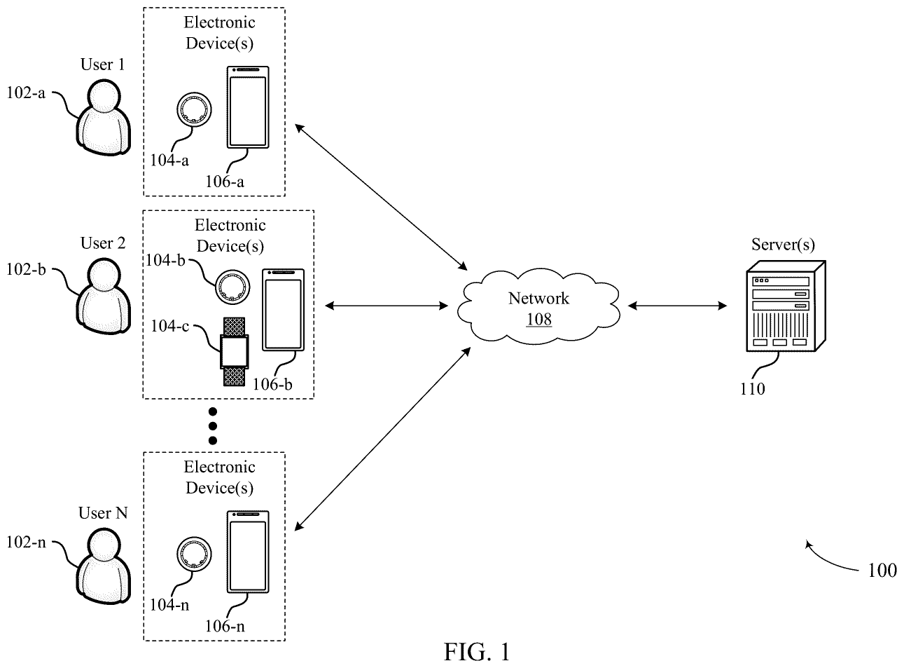

FIG. 1 illustrates an example of a system 100 that supports a wearable ring device with deformable inner cover in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state: 2) circadian rhythms: 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used): 5) multi-day ovarian rhythms in women and spermatogenesis rhythms in men: 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for implementing a wearable ring device (e.g., wearable device 104) that uses a material on the inner surface of the wearable device such that the finger may change in size without affecting the fit of the wearable device. The wearable ring device 104 may include a sensor module comprising one or more sensors configured to acquire physiological data from a user and a deformable material that extends along one or more portions of the inner circumferential surface of the wearable device.

The deformable material may be arranged to enhance a signal quality of measurements by the one or more sensors as well as avoid interfering with the measurements by the one or more sensors. For example, the deformable material may be configured to exert pressure on the finger by conforming to an outer circumferential surface of the finger. In such cases, the deformable material may form an interference fit with the finger of the user when the wearable ring device is worn by the user. The fit of the ring may be improved through the use of a deformable material that allows the wearable ring device to adapt to the anatomy of the finger. In such cases, by including a deformable material in the inner circumferential surface of the wearable ring device, the wearable ring device may acquire more accurate physiological data measurements based on the personalized, improved fit, thereby increasing the efficiency and accuracy of the signal as compared to other conventional rings (e.g., jewelry). As described in more detail herein, the deformable material may be arranged and sized to avoid interfering with the measurements obtained from the one or more sensors. This is a design consideration that is not present in other ring-type devices (e.g., jewelry or other wearables that do not contain sensors). As such, the deformable material not only enhances the fit of the wearable ring device (and thereby enhances the measurements themselves), but also avoids interfering with the measurements by not blocking or distorting light or other measured signals through or around the deformable material.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

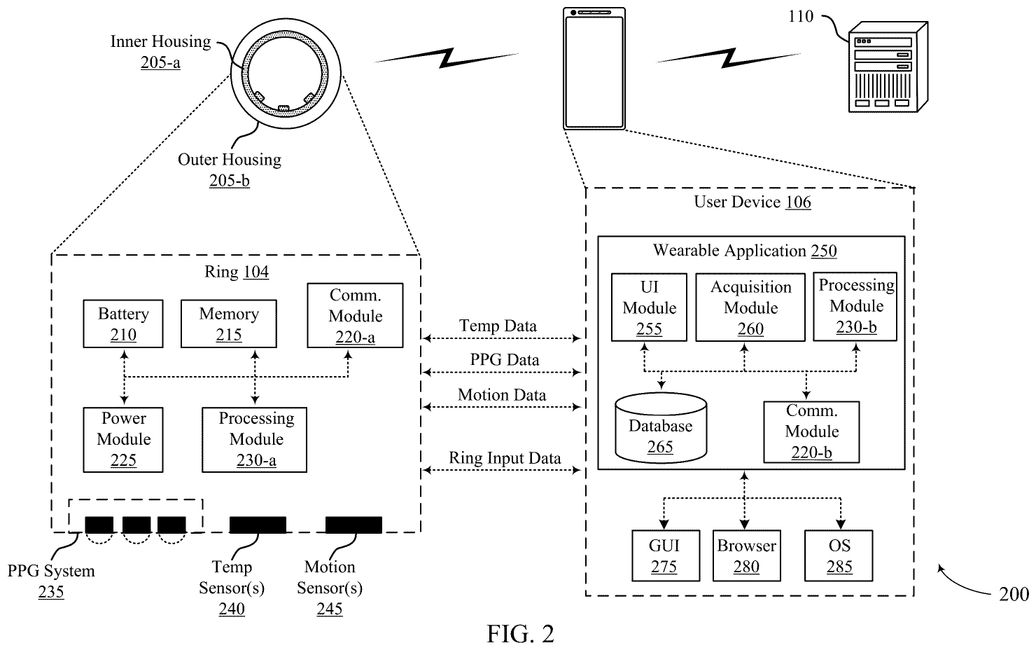

FIG. 2 illustrates an example of a system 200 that supports a wearable ring device with deformable inner cover in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency.” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support a wearable device that improves the fit of the wearable device by using a material on the inner surface of the wearable device that conforms to the user. In particular, techniques described herein support a ring 104, such as a wearable device 104 as described with reference to FIG. 1. For example, a ring 104 may include an inner housing 205-a configured to house a sensor module that includes one or more sensors that are configured to acquire physiological data from a user 102. The one or more sensors of the ring 104 may obtain physiological measurements from the user (e.g., temperature sensors, additional LED-PD sensors used for measuring heart rate, oxygen saturation, one or more sensors that a device may use to detect whether a user is asleep, or the like).

In some cases, the one or more sensors of the ring 104 are configured to acquire the physiological data from the user based on arterial blood flow, temperature, etc. In some implementations, the one or more sensors of the ring 104 are configured to acquire the physiological data (e.g., including PPG data) from the user based on blood flow that is diffused into the microvascular bed of skin with capillaries and arterioles. The one or more sensors of the ring 104 may be an example of photodetectors from the PPG system 235, temperature sensors 240, motion sensors 245, galvanic sensors, and other sensors.

As described herein, the wearable device of the system 200 may include a deformable material that extends along one or more portions of the inner circumferential surface of the housing (e.g., inner housing 205-a). The deformable material is configured to exert pressure on a finger of the user by conforming to an outer circumferential surface of the finger to form an interference fit with the finger of the user. The deformable material may be arranged to enhance a signal quality of measurements by the one or more sensors while avoiding interfering with the measurements by the one or more sensors.

While much of the present disclosure describes one or more components in the context of a wearable ring device, aspects of the present disclosure may additionally or alternatively be implemented in the context of other wearable devices. For example, in some implementations, the one or more components described herein may be implemented in the context of other wearable devices, such as bracelets, watches, necklaces, piercings, and the like. For example, the wearable device 104 may surround a finger, wrist, ankle, earlobe, or the like of a user.

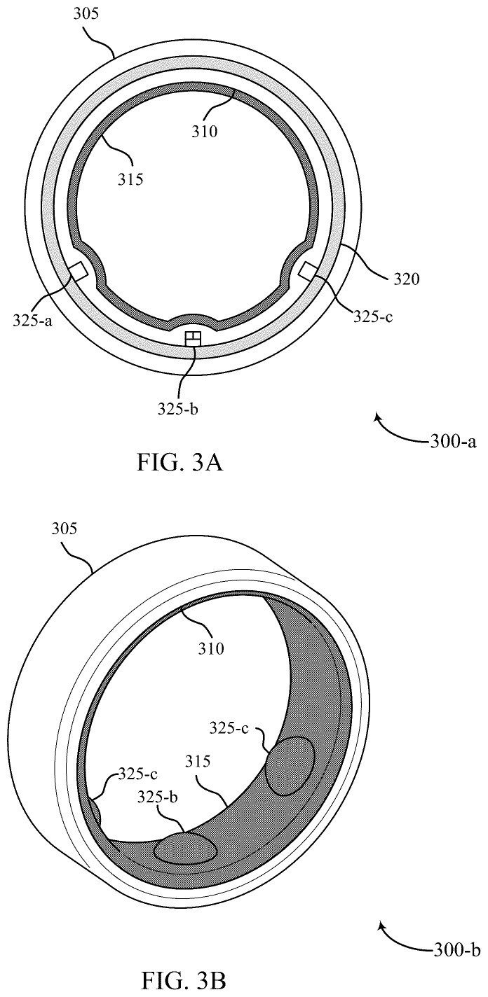

FIG. 3A shows an example of a side view of a wearable ring device 300-a that supports a wearable ring device with deformable inner cover in accordance with aspects of the present disclosure. The wearable ring device 300-a may implement, or be implemented by, aspects of the system 100, system 200, or both. For example, the wearable ring device 300-a may illustrate examples of a wearable ring device with a deformable material as described with reference to FIGS. 1 and 2.

The wearable ring device 300-a may include a housing 305 that includes an inner shell component and an outer shell component, which may be examples of an inner housing and an outer housing, respectively, as described with reference to FIG. 2. The outer shell component may be an example of a ring outer cover. The outer shell component may include an outer circumferential surface and an inner circumferential surface 310 opposite the outer circumferential surface.

The wearable ring device 300-a may include an electronic substrate, such as a printed wiring board (PWB) or PCB 320. The PCB 320 may have both flexible and rigid sections. In some cases, the PCB 320 may include flexible sections that extend around an entire circumference of the wearable ring device 300-a. Electrical components may be embedded in the PCB 320 of the wearable ring device 300-a. The electrical components of the wearable ring device 300-a may include one or more sensors 325 (e.g., temperature sensors, galvanic sensors, light sources, photodetectors) configured to acquire physiological data associated with the user. The one or more sensors 325 of the wearable ring device 300-a may be positioned at least partially within the housing 305 of the wearable ring device 300-a. For example, the wearable ring device 300-a may include a sensor 325-a, a sensor 325-b, and a sensor 325-c, where the sensors 325 may be embedded within the housing 305.

The sensors 325 may be configured to acquire physiological data associated with the user. In such cases, the wearable ring device 300-a may collect data, such as physiological data, from a user that can be used to determine health and wellness information for the user. For example, the wearable ring device 300-a may collect the data via the one or more sensors 325 that couple with (e.g., interface with, contact) the skin of the user. The reliability (e.g., accuracy) of the data collected by the wearable ring device 300-a may be contact-dependent, meaning that the reliability may vary with the contact between the wearable ring device 300-a and the skin of the user. But the contact between the wearable ring device 300-a and the skin of the user may be inconsistent (e.g., due to changes in finger position, changes in finger girth, differences in finger shapes, and the like).

In some cases, skin contact between the user's finger and the wearable ring device 300-a may vary due to an external force pushing on the wearable ring device 300-a, a gap between the user's finger and the wearable ring device 300-a, liquid or contaminants between the user's finger and the wearable ring device 300-a, a poorly-fitting wearable ring device 300-a, or a combination thereof. Changes in skin contact and/or pressure against the user's finger may affect physiological measurements. For example, the changes in skin contact and/or pressure may cause inaccuracy in the physiological measurements and higher battery consumption. In some examples, the wearable ring device 300-a may move around the user's finger, causing rotation of the wearable ring device 300-a, and thereby losing contact between one or more sensors 325 that may affect the physiological measurements.

Use of unreliable data collected while the contact between the wearable ring device 300-a is poor may result in inaccurate health and wellness information for the user. For example, using data while the contact between the wearable ring device 300-a and the user changes may result inaccurate physiological data readings, which may lead to a distorted picture of the user's overall health, as well as increased power consumption and decreased battery life.

Accordingly, to facilitate improved physiological measurements, aspects of the present disclosure are directed to including a deformable material 315 along one or more portions of an inner circumferential surface 310 of the housing 305. The deformable material 315 may allow the finger to grow or reduce in size while maintaining contact between the finger and the wearable ring device 300-a. By using a memory shape polymer material that changes with skin or body temperature, a soft polymer material that changes with external force, or both, the fit of the wearable ring device 300-a may be improved. In such cases, the wearable ring device 300-a may maintain an accurate fit as the finger girth, shape, or circumference changes over time. For example, integrating a deformable material 315 into the inner circumference of the wearable ring device 300-a may lead to more accurate physiological data measurements as the contact between the wearable ring device 300-a and the user is improved, thereby increasing the efficiency and accuracy of the signal.

The deformable material 315 may extend along the inner circumferential surface 310 of the housing 305. In some cases, the deformable material 315 may extend along the entire circumference of the inner circumferential surface 310. In such cases, the deformable material 315 may extend three hundred and sixty degrees around the inner circumferential surface 310 relative to an axis of the housing 305. The deformable material 315 may extend over the one or more sensors 325. For example, the deformable material 315 may be disposed on top of the one or more sensors 325 such that the deformable material 315 is positioned at a same location as the one or more sensors 325. The deformable material 315 may extend to a predetermined thickness from the inner circumferential surface 310.

As the deformable material 315 extends around the entire circumference of the inner circumferential surface 310 of the housing 305, the deformable material 315 is disposed along the one or more portions of the inner circumferential surface 310 adjacent to the one or more sensors 325 and along the one or more portions of the inner circumferential surface 310 opposite the sensors 325. The one or more sensors 325 may be configured to perform measurements through the deformable material 315.

The deformable material 315 may be arranged to avoid interfering with measurements by the one or more sensors 325. For example, the deformable material 315 may include optical properties that propagate light from the one or more sensors 325 into the finger of the user. Additionally or alternatively, the deformable material 315 may be sized and arranged with respect to the one or more sensors 325 such that even in a deformed state, the deformable material 315 will not interfere with the measurements by the one or more sensors 325. The deformable material 315 may enhance the signal quality by allowing light emitted from the sensor 325-b to propagate through the deformable material 315 and into the tissue of the user. In some cases, the deformable material 315 may be configured to interface with the housing 305 such that one or more antenna elements disposed within the wearable ring device 300-a wirelessly couple one or more components of the wearable ring device 300-a with a user device, thereby decreasing an amount of noise in the signal and increasing the efficiency and accuracy of the signal.

In some examples, the deformable material 315 may be arranged to enhance a signal quality of measurements by the one or more sensors 325 through the application of pressure on the finger of the user within an area of the finger adjacent to the one or more sensors 325. For example, as the deformable material 315 extends around the entire circumference of the wearable ring device 300-a (e.g., including over the sensors 325), the sensors 325 may contact the finger of the user, thereby applying pressure to the finger. In some cases, the deformable material 315 may enhance the signal quality of measurements by the one or more sensors 325 through the application of pressure on the finger within an entire surface area of the deformable material 315 (e.g., around an entire circumference of the wearable ring device 300-b).

The deformable material 315 may be elastically deformable such that the deformable material 315 contracts to form the interference fit around the digit of the user when the wearable ring device 300-a is worn by the user and the deformable material 315 expands when the wearable ring device 300-a is off of the digit of the user. In such cases, the deformable material 315 may be an example of a soft, polymer material that responds to mechanical stress on the deformable material 315.

In some cases, the deformable material 315 may include, but is not limited to, polyurethane, silicon, a flexible material, a chemical-resistant material, a memory shape polymer material, a soft polymer material, a thermally resistant material, or a combination thereof. In some cases, the deformable material 315 may be an example of a transparent material configured to transmit light through the deformable material 315. The memory shape polymer material of the deformable material 315 may change from an undeformed state and to a deformed state when induced by an external stimulus (e.g., temperature change). In such cases, the memory shape polymer material may include an ability to transition from a deformed state to the undeformed state after the external stimulus is removed.

For example, an elasticity of the deformable material 315 may change with temperature to change a shape, a thickness, or both of the deformable material 315 when the wearable ring device 300-a is worn by the user. In such cases, the deformable material 315 may be an example of a memory shape polymer that deforms at the body temperature of the user. For example, the glass transition temperature of the deformable material 315 may be a same temperature as the body temperature (e.g., the temperature of the skin surface). In some examples, the deformable material 315 may include a thermal resistant material such that the deformable material 315 may become deformable at the body temperature of the user (e.g., when the wearable ring device 300-a is placed on the finger of the user) to enable the deformable material 315 to be molded around the finger.

In some cases, the deformable material 315 may include a material with a high surface friction such that friction between the deformable material 315 and the user's finger may not affect the deformable material 315 and the measurements by the one or more sensors 325. The deformable material 315 may include a chemical resistance material such that the deformable material 315 may not react with oils, lotions, sweat, water, and the like and avoid absorbing the materials into the deformable material 315. A material (e.g., oils, creams, sweat, water, and the like) between the sensors and the finger of the user may dampen or absorb the light, resulting in less light reaching the sensors 325.

FIG. 3B shows an example of a partial top down view of a wearable ring device 300-b that supports a wearable ring device with deformable inner cover in accordance with aspects of the present disclosure. In some cases, the inner circumferential surface 310 may be coated with the deformable material 315 such that the deformable material 315 may be adhered to the inner circumferential surface 310 of the housing 305. For example, the deformable material may be adhered to a hard, non-deformable polymer material of the inner circumferential surface 310. In some cases, the deformable material 315 may be molded into the inner circumferential surface 310 of the housing 305. In some cases, the domes over the sensors 325 may include the deformable material 315. In such cases, the deformable material 315 may be adhered to the transparent material of the domes (e.g., sensors 325). In some cases, the one or more materials may be positioned between the sensors 325 and the deformable material 315.

The deformable material 315 may be configured to form around the outer circumferential surface of the finger of the user to eliminate air gaps between the outer circumferential surface of the finger and the inner circumferential surface 310 of the housing 305. In such cases, the deformable material 315 enhances the signal quality by eliminating the air gap and creating a tighter fit between the finger and the wearable ring device 300-b. An airgap between the skin of the user and the wearable ring device 300-b may disturb the optical path (e.g., because the light is now coupled to the finger through an additional interface) such that some or all of the light is directed away from the sensors 325. In such cases, the deformable material 315 may be disposed around the entire circumference of the wearable ring device 300-b to exert pressure on the finger around an entire circumference of the finger by conforming to the outer circumferential surface of the finger and forming an interference fit with the finger when the wearable ring device 300-b is worn by the user.

The deformable material 315 of the inner circumferential surface 310 may be configured to interact with surface of the finger of the user to fit the wearable ring device 300-b onto the finger of the user. As the wearable ring device 300-b is positioned onto the finger of the user, the deformable material 315 may conform to an outer circumferential surface of the finger to form an interference fit with the finger when the wearable ring device 300-b is worn. For example, the deformable material 315 may expand to fill the gaps between the wearable ring device 300-b and the finger of the user such that the deformable material 315 contacts the outer circumferential surface of the finger. In such cases, the deformable material 315 may be an example of a memory shape polymer material where the elasticity changes with skin or body temperature such that the deformable material 315 conforms to the finger.

In other examples, the deformable material 315 may be pressed inwards towards the inner circumferential surface 310 as the wearable ring device 300-b is placed on the finger such that the deformable material 315 contracts to fit the finger of the user. The deformable material 315 may exert a pressure on the finger on the user within an area of the finger adjacent to the sensors 330 as well as an area of the finger contacting the deformable material 315 (e.g., the entire circumference of the wearable ring device 300-b). In such cases, the deformable material 315 may provide ample contact between the sensors 330 and the finger, thereby resulting in accurate physiological measurements and enhancing the signal quality of measurements.

In some cases, the deformable material 315 may be manufactured onto the wearable ring device 300-b by using a flexible silicon mold, a non-deformable, polymer mold, or both. The mold may include a metal core to create the inner circumferential surface 310. The deformable material 315 may be desisted onto the inner circumferential surface 310 after the wearable ring device 300-b is manufactured.

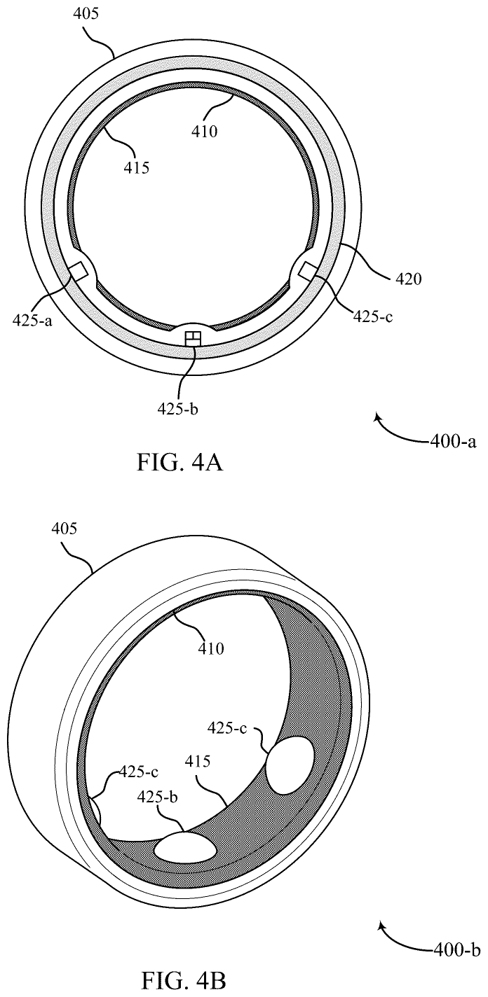

FIG. 4A shows an example of a side view of a wearable ring device 400-a that supports a wearable ring device with deformable inner cover in accordance with aspects of the present disclosure. The wearable ring device 400-a may implement, or be implemented by, aspects of the system 100, system 200, or both. For example, the wearable ring device 400-a may illustrate examples of a wearable ring device as described with reference to FIGS. 1-3.

As described with reference to FIG. 3, the wearable ring device 400-a may be an example of the wearable ring device 300-a. The wearable ring device 400-a may include housing 405, inner circumferential surface 410, PCB 420, and sensors 425. The wearable ring device 400-a may include deformable material 415. The deformable material 415 may be an example of the deformable material 315, as described with reference to FIG. 3.

The deformable material 415 may be disposed along one or more portions of the inner circumferential surface 410 of the housing 405. For example, the deformable material 415 may be disposed opposite of the one or more sensors 425 and adjacent to the one or more sensors 425. The deformable material 415 positioned opposite of the sensors 425 may be included on a top portion of the inner circumferential surface 410 while the sensors 425 may be position on a bottom portion of the inner circumferential surface 410. The deformable material 415 may not be disposed over the one or more sensors 425. In such cases, the domes over the one or more sensors 425 may not include the deformable material 415. The deformable material 415 may extend to a thickness from the inner circumferential surface 410 that aligns with a height of the domes to provide a smooth, continuous surface around the inner circumferential surface 410 of the housing 405.

The deformable material 415 may be arranged to avoid interfering with measurements by the one or more sensors 425 such that the deformable material 415 may not be disposed over the sensors 425. In some examples, the deformable material 415 may be arranged to enhance a signal quality of measurements by the one or more sensors 425 through the application of pressure on the finger of the user within an area of the finger adjacent to the one or more sensors 425. For example, by disposing the deformable material 415 around the inner circumferential surface 410 except for over the sensors 425, the sensors 425 may directly contact the finger of the user and apply pressure to the finger adjacent to the one or more sensors 425.

FIG. 4B shows an example of a partial top down view of a wearable ring device 400-b that supports a wearable ring device with deformable inner cover in accordance with aspects of the present disclosure. In some cases, the inner circumferential surface 410 may be coated with the deformable material 415 such that the deformable material 415 may be adhered to the inner circumferential surface 410 of the housing 405, the deformable material 415 may be molded into the inner circumferential surface 410, or both.

With reference to FIG. 4B, portions of the deformable material 415 may extend around the entire circumference of the wearable ring device 400-b. For example, the outermost edges of the inner circumferential surface 410 may be lined with the deformable material 415, while the deformable material 415 may be excluded from the portions over the one or more sensors 425. In such cases, the deformable material 415 may extend three hundred and sixty degrees around the inner circumferential surface 410 relative to an axis of the housing 405 in some portions of the wearable ring device 400-b whereas the deformable material 415 may extend less than three hundred and sixty degrees around the inner circumferential surface 410 relative to an axis of the housing 405 in other portions of the wearable ring device 400-b (e.g., in a location where the sensors 425 are located).

The deformable material 415 may be configured to form around the sensors 425 and the outer circumferential surface of the finger of the user to eliminate air gaps between the outer circumferential surface of the finger and the inner circumferential surface 410 of the housing 405. In some cases, the deformable material 415 disposed around the sensors 425 may direct light from the sensor 425-b and into the tissue and then direct the light into the sensors 425-a and 425-c.

The wearable ring device 400-b may be manufactured by depositing the deformable material 415 onto the inner circumferential surface 410. For example, the deformable material 415 may be disposed on the hard, non-deformable polymer material of the inner circumferential surface 410. The deformable material 415 may be removed from over the sensors 425 such that the deformable material 415 may include cut outs over the sensors 425. The sensors 425 may include a transparent, non-deformable material over then. In some cases, the deformable material 415 circumscribing the domes over the sensors 425 may include a light blocking material such that light may be directed out of the sensors 425 and into the tissue of the user.

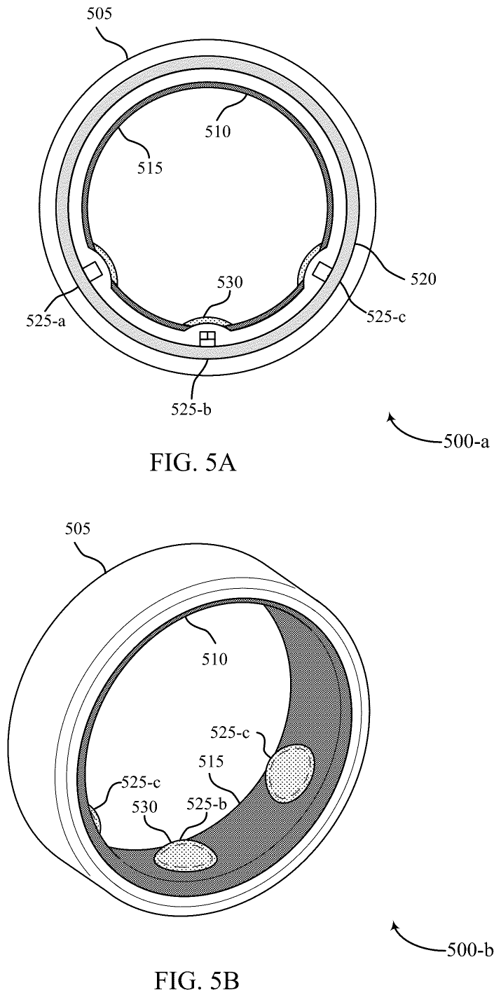

FIG. 5A shows an example of a side view of a wearable ring device 500-a that supports a wearable ring device with deformable inner cover in accordance with aspects of the present disclosure. The wearable ring device 500-a may implement, or be implemented by, aspects of the system 100, system 200, or both. For example, the wearable ring device 500-a may illustrate examples of a wearable ring device as described with reference to FIGS. 1-4.

As described with reference to FIGS. 3 and 4, the wearable ring device 500-a may include housing 505, inner circumferential surface 510, PCB 520, and sensors 525. The wearable ring device 500-a may include deformable material 515. The deformable material 515 may be an example of the deformable material, as described with reference to FIGS. 3 and 4. As described with reference to FIG. 4, the deformable material 515 may be manufactured and positioned in a same manner as the deformable material 415.

The wearable ring device 500-a may include a material 530 disposed over the sensors 525. The material 530 may be an example of a second deformable material, as described with reference to FIGS. 3 and 4. In other examples, the material 530 may be an example of a layer of material disposed over the sensors 525 that is a different material than the deformable material 515.

The material 530 may be disposed along one or more portions of the inner circumferential surface 510 of the housing 505. For example, the material 530 may be disposed at a same location as the sensors 525. The material 530 may be disposed on top of the domes. In such cases, the material 530 may be included on the domes in a bottom portion of the inner circumferential surface 510. The material 530 may include a same thickness as the deformable material 515. In such cases, the deformable material 515 may extend to a same thickness from the inner circumferential surface 510 as the material 530.

The material 515 may be arranged to avoid interfering with measurements by the one or more sensors 525 such that material 530 may be arranged to enhance a signal quality of measurements by the one or more sensors 525 through the application of pressure on the finger of the user within an area of the finger adjacent to the one or more sensors 525. For example, by disposing the material 530 around the inner circumferential surface 510 over the sensors 525, the sensors 525 may directly contact the finger of the user and apply pressure to the finger. For example, the material 530 may include optical properties that propagate light from the one or more sensors 525 into the finger of the user such that the light emitted from the sensor 525-b may propagate through the material 530 and into the tissue of the user.

The material 530 may be an example of a transparent material configured to transmit light through the material 530. The material 530 may be elastically deformable such that the material 530 contracts to form the interference fit around the finger of the user when the wearable ring device 500-a is worn by the user and expands when the wearable ring device 500-a is off of the finger of the user. In such cases, the material 530 may be an example of a soft, polymer material that responds to mechanical stress.

In some cases, the material 530 may include, but is not limited to, polyurethane, silicon, a flexible material, a chemical-resistant material, a memory shape polymer material, a soft polymer material, a thermally resistant material, or a combination thereof. The memory shape polymer material may change from an undeformed state and to a deformed state when induced by an external stimulus (e.g., temperature change). In such cases, the memory shape polymer material of the material 530 may include an ability to transition from a deformed state to the undeformed state after the external stimulus is removed. In some cases, the material 530 may be an example of a hard, non-deformable polymer material. In such cases, the inner circumferential surface 510 may include portions of deformable material 515 and portions of material 530.

FIG. 5B shows an example of a partial top down view of a wearable ring device 500-b that supports a wearable ring device with deformable inner cover in accordance with aspects of the present disclosure. In some cases, the inner circumferential surface 510 may be coated with the deformable material 515 such that the deformable material 515 may be adhered to the inner circumferential surface 510 of the housing 505, the deformable material 515 may be molded into the inner circumferential surface 510, or both.

The domes may be coated with the material 530 such that the material 530 may be adhered to the inner circumferential surface 510 of the housing 505 over the sensors 525, the material 530 may be molded into the inner circumferential surface 510 over the sensors 525, or both. The material 530 may be configured to conform to the outer circumferential surface of the finger of the user to eliminate air gaps between the outer circumferential surface of the finger and the inner circumferential surface 510 of the housing 505 (e.g., including the one or more sensors 525). In some cases, the material 530 disposed over the sensors 525 may direct light from the sensor 525-b and into the tissue and then direct the light into the sensors 525-a and 525-c.

The wearable ring device 500-b may be manufactured by depositing the deformable material 515 onto the inner circumferential surface 510. The deformable material 515 may be removed from over the sensors 525 such that the deformable material 515 may include cut outs over the sensors 525. The material 530 may then be deposited over the sensors 525. In some cases, the material 515 deposited on the domes over the sensors 525 may include a light blocking material such that light may be directed out of the sensors 525 and into the tissue of the user.

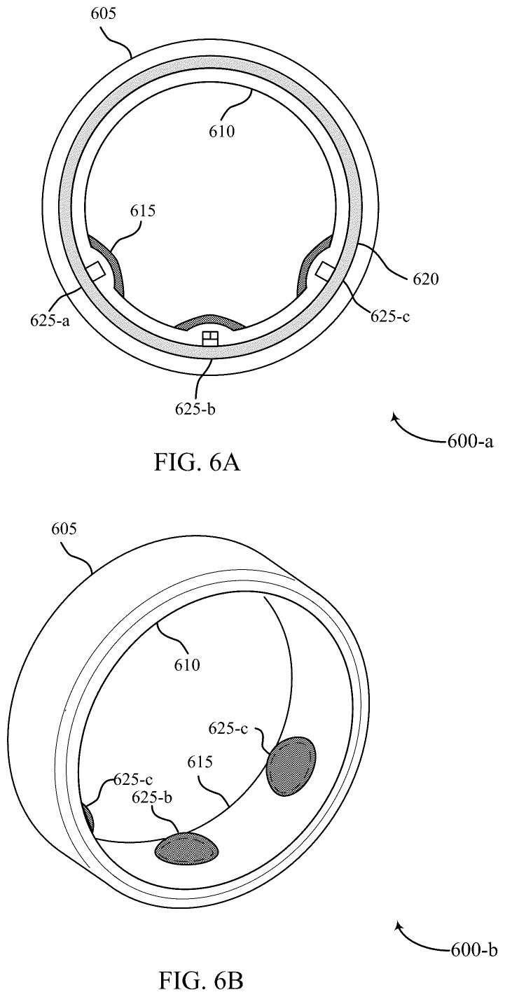

FIG. 6A shows an example of a side view of a wearable ring device 600-a that supports a wearable ring device with deformable inner cover in accordance with aspects of the present disclosure. The wearable ring device 600-a may implement, or be implemented by, aspects of the system 100, system 200, or both. For example, the wearable ring device 600-a may illustrate examples of a wearable ring device as described with reference to FIGS. 1-5.

As described with reference to FIGS. 3-5, the wearable ring device 600-a may include housing 605, inner circumferential surface 610, PCB 620, and sensors 625. The wearable ring device 600-a may include deformable material 615. The deformable material 615 may be an example of the deformable material, as described with reference to FIGS. 3-5.

The deformable material 615 may be disposed over the sensors 625. For example, the deformable material 615 may be disposed at a same location as the sensors 625. The deformable material 615 may be disposed on top of the domes. In such cases, the deformable material 615 may be included on the domes in a bottom portion of the inner circumferential surface 610. The deformable material 615 may extend to a thickness from the inner circumferential surface 610 as to cushion and/or apply pressure to the skin tissue of the user. In some cases, an additional layer of material may be disposed between the sensors 625 and the deformable material 615. As described with reference to FIG. 3, the deformable material 615 may be arranged to avoid interfering with measurements by the one or more sensors 625 to enhance the signal quality of measurements by the sensor 625.

The remaining portions of the inner circumferential surface 610 (e.g., portions without the deformable material 615) may include a hard material of the inner circumferential surface 610. In such cases, the hard material may extend along one or more portions of the inner circumferential surface 610 of the housing 605. The hard material may be an example of a non-deformable material such as a metallic inlet (e.g., a metallic material), a ceramic material, a hard polymer material, or a combination thereof. The deformable material 615 may be disposed adjacent to the one or more portions of the hard material. For example, the hard material may circumscribe the sensors 625 where the deformable material 615 is adhered to.

FIG. 6B shows an example of a partial top down view of a wearable ring device 600-b that supports a wearable ring device with deformable inner cover in accordance with aspects of the present disclosure. In some cases, the inner circumferential surface 610 may be coated with the deformable material 615 such that the deformable material 615 may be adhered to the inner circumferential surface 610 of the housing 605 over the sensors 625. For example, the deformable material 615 may be adhered to a transparent material of the inner circumferential surface 610. In some cases, the deformable material 615 may be molded into the inner circumferential surface 610 of the housing 605.

The deformable material 615 may be configured to apply pressure between the user's skin and the sensors 625 to eliminate air gaps between the outer circumferential surface of the finger and the sensors 625. In such cases, the deformable material 615 enhances the signal quality by eliminating the air gap and creating a tighter fit between the finger and the wearable ring device 600-b (e.g., the sensors 625). The deformable material 615 disposed over the sensors 625 may form an interference fit between the sensors 625 of the wearable ring device 600-b and the finger when the wearable ring device 600-b is worn by the user.

For example, the deformable material 615 may expand to fill the gaps between the wearable ring device 600-b and the finger of the user such that the deformable material 615 contacts the outer circumferential surface of the finger. In such cases, the deformable material 615 may be an example of a memory shape polymer material where the elasticity changes with skin or body temperature such that the deformable material 615 changes to conform to the finger. In other examples, the deformable material 615 may be pressed inwards towards the inner circumferential surface 610 as the wearable ring device 600-b is placed on the finger such that the deformable material 615 contracts to fit the finger of the user and maintains the wearable ring device 600-b in a fixed position on the user's finger. The deformable material 615 may exert a pressure on the finger on the user within an area of the finger on top of and adjacent to the sensors 625. In such cases, the deformable material 615 may provide ample contact between the sensors 625 and the finger, thereby resulting in accurate physiological measurements and enhancing the signal quality of measurements.

In some cases, the deformable material 615 may be manufactured onto the wearable ring device 600-b depositing the deformable material 615 onto the domes of the sensors 625 after the wearable ring device 600-b is manufactured. For example, the deformable material 615 may be deposited on top of the domes and cut out to the fit over the sensors 625. In some cases, the deformable material 615 may be coupled to the hard material of the inner circumferential surface 610 to form a seal of the deformable material 615 over and around the sensors 625.

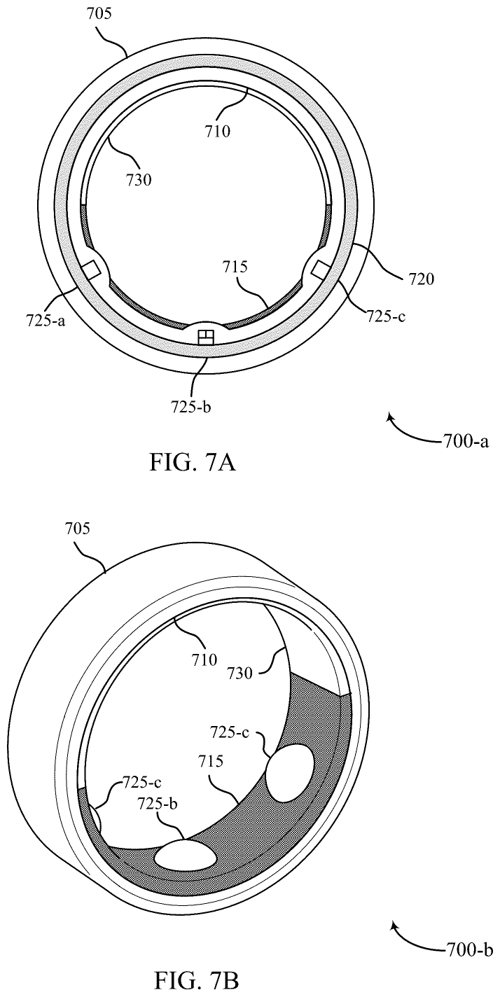

FIG. 7A shows an example of a side view of a wearable ring device 700-a that supports a wearable ring device with deformable inner cover in accordance with aspects of the present disclosure. The wearable ring device 700-a may implement, or be implemented by, aspects of the system 100, system 200, or both. For example, the wearable ring device 700-a may illustrate examples of a wearable ring device as described with reference to FIGS. 1-6.

As described with reference to FIGS. 3-6, the wearable ring device 700-a may include housing 705, inner circumferential surface 710, PCB 720, and sensors 725. The wearable ring device 700-a may include deformable material 715. The deformable material 715 may be an example of the deformable material, as described with reference to FIGS. 3-6.

The deformable material 715 may be disposed along one or more portions of the inner circumferential surface 710 of the housing 705. For example, the deformable material 715 may be disposed adjacent to the one or more sensors 725 such that the deformable material 715 surrounds the sensors 725 but is not disposed on top of the sensors 725. In such cases, the domes over the one or more sensors 725 may not include the deformable material 715. The deformable material 715 may extend to a thickness from the inner circumferential surface 710 that aligns with a height of the domes.

The deformable material 715 may be arranged to avoid interfering with measurements by the one or more sensors 725 such that the deformable material 715 may not be disposed over the sensors 725. In some examples, the deformable material 715 may be arranged to enhance a signal quality of measurements by the one or more sensors 725 through the application of pressure on the finger of the user within an area of the finger adjacent to the one or more sensors 725. For example, by disposing the deformable material 715 around the inner circumferential surface 710 except for over the sensors 725, the sensors 725 may directly contact the finger of the user and apply pressure to the finger.

The wearable ring device 700-a may include a material 730 disposed on the top portion of the inner circumferential surface 710 of the housing 705. The material 730 may extend along one hundred eighty degrees of the top portion relative to an axis of the housing 705. The material 730 may be an example of the material 530, as described with reference to FIG. 5. For example, the material 730 may be an example of a hard material or a second deformable material. In such cases, the material 730 may include polyurethane, silicon, a flexible material, a chemical-resistant material, a memory shape polymer material, a soft polymer material, a thermally resistant material, or a combination thereof. The material 730 may be different than the deformable material 715.

By including the material 730 on the top portion of the inner circumferential surface 710, the material 730 may be arranged to avoid interfering with measurements by the one or more sensors 725 such that material 730 may be arranged to enhance a signal quality of measurements by the one or more sensors 725 through the application of pressure on the finger of the user within an area of the finger opposite of the one or more sensors 725.

The wearable ring device 700-a may include a combination of a memory shape polymer material and a soft, polymer material. For example, one of the material 730 or the deformable material 715 may be elastically deformable such that the material 730 or the deformable material 715 contracts to form the interference fit around the digit of the user when the wearable ring device 700-a is worn by the user. After the wearable ring device 700-a is removed from the finger, the material 730 or the deformable material 715 expands back to the original, undeformed state. In such cases, the material 730 or the deformable material 715 may be an example of a soft, polymer material that responds to mechanical stress. In other examples, the other of the material 730 or deformable material 715 may be an example of the memory shape polymer material that transitions from an undeformed state to a deformed state after the external stimulus (e.g., being placed on the finger of the user) is applied.

FIG. 7B shows an example of a partial top down view of a wearable ring device 700-b that supports a wearable ring device with deformable inner cover in accordance with aspects of the present disclosure. In some cases, the inner circumferential surface 710 may be coated with the deformable material 715 and the material 730 such that the deformable material 715 and material 730 may be adhered to the inner circumferential surface 710 of the housing 705. In some examples, the deformable material 715 and the material 730 may be molded into the inner circumferential surface 710.

The deformable material 715 may be configured to form around the sensors 725 and the outer circumferential surface of the finger of the user to eliminate air gaps between the outer circumferential surface of the finger and the inner circumferential surface 710 of the housing 705. In some cases, the deformable material 715 disposed around the sensors 725 may direct light from the sensor 725-b and into the tissue and then direct the light into the sensors 725-a and 725-c.

The wearable ring device 700-b may be manufactured by depositing the deformable material 715 onto the inner circumferential surface 710. For example, the deformable material 715 may be disposed on the material of the inner circumferential surface 710. The deformable material 715 may be removed from over the sensors 725 such that the deformable material 715 may include cut outs over the sensors 725. In some cases, the deformable material 715 surrounding the sensors 725 may include a light blocking material such that light may be directed out of the sensors 725 and into the tissue of the user.

The wearable ring device 700-b may be additionally manufactured by depositing the material 730 onto the inner circumferential surface 710 along the top, inner portion of the wearable ring device 700-b opposite of the sensors 725. For example, the material 730 may be disposed on top of the inner circumferential surface 710 extending one hundred and eighty degrees relative to the axis of the housing 705. In some examples, the material 730 and the deformable material 715 may be swapped such that the deformable material 715 may be adhered to the top, inner portion of the inner circumferential surface 710, and the material 730 may be deposited on the bottom, inner portion of the inner circumferential surface 710.

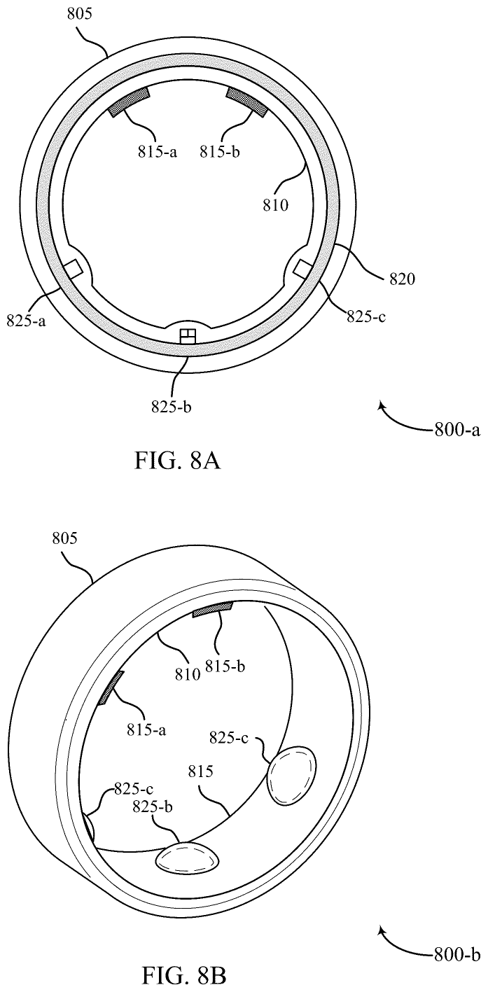

FIG. 8A shows an example of a side view of a wearable ring device 800 that supports a wearable ring device with deformable inner cover in accordance with aspects of the present disclosure. The wearable ring device 800-a may implement, or be implemented by, aspects of the system 100, system 200, or both. For example, the wearable ring device 800-a may illustrate examples of a wearable ring device as described with reference to FIGS. 1-7.

As described with reference to FIGS. 3-7, the wearable ring device 800-a may include housing 805, inner circumferential surface 810, PCB 820, and sensors 825. The wearable ring device 800-a may include deformable material 815. The deformable material 815 may be an example of the deformable material, as described with reference to FIGS. 3-7.

The deformable material 815 may be an example of pads and/or patches of deformable material 815 that extend along one or more portions of the inner circumferential surface 810. The pads of deformable material 815 may be positioned at various positions around the inner circumferential surface 810. In some cases, the inner circumferential surface 810 may include a single pad of deformable material 815, a first deformable material 815-a and a second deformable material 815-b, or any combination of a quantity of pads of deformable material 815. As described with reference to FIG. 8A, the wearable ring device 800-a may include pads of deformable material 815 located on an opposite side of the inner circumferential surface 810. In such cases, the pads of deformable material 815 may be located on a top portion of the inner circumferential surface 810 and the sensors 825 may be located on a bottom portion of the inner circumferential surface 810.

The deformable material 815 may be disposed on a surface of the hard material of the inner circumferential surface 810. In some cases, the deformable material 815 may be disposed adjacent to the one or more portions of the hard material. The deformable material 815 may extend a thickness away from the surface of the inner circumferential surface 810 such that the deformable material 815 may contact the tissue surface of the user and provide pressure opposite of the sensors 825.

The deformable material 815 may be arranged to avoid interfering with measurements by the one or more sensors 825 such that the deformable material 815 may not be disposed over the sensors 825. In some examples, the deformable material 815 may be arranged to enhance a signal quality of measurements by the one or more sensors 825 through the application of pressure on the finger of the user within an area of the finger adjacent to the one or more sensors 825 and an area opposite of the sensors 825. For example, by disposing the deformable material 815 on the inner circumferential surface 810 opposite of the sensors 825, the sensors 825 may directly contact the finger of the user and apply pressure to the finger.

The deformable material 815 may be an example of a heat-activated material that may grow and expand after the wearable ring device 800-a is placed on the finger as the temperature of the deformable material 815 changes due the user's skin temperature. The deformable material 815 may expand to contact the outer circumferential surface of the user's finger after the wearable ring device 300-a is worn by the user. The deformable material 815 positioned on the inner circumferential surface 810 may create a tighter seal and tighter fit of the wearable ring device 800-a.

FIG. 8B shows an example of a partial top down view of a wearable ring device 800-b that supports a wearable ring device with deformable inner cover in accordance with aspects of the present disclosure. The wearable ring device 800-b may be manufactured by depositing the deformable material 815 onto the inner circumferential surface 810. For example, the deformable material 815 may be disposed on top of the hard, non-deformable polymer material of the inner circumferential surface 810, as described with reference to FIGS. 3-7.

It should be noted that the apparatuses described above describe possible implementations, and that the operations and the steps may be rearranged or otherwise modified and that other implementations are possible. Furthermore, aspects from two or more of the apparatuses may be combined.

An apparatus is described. The apparatus may include a housing comprising one or more sensors configured to acquire physiological data from a user and a deformable material extend along one or more portions of an inner circumferential surface of the housing and configured to exert pressure on a digit of the user by conforming to an outer circumferential surface of the digit to form an interference fit with the digit of the user when the wearable ring device is worn by the user, wherein the deformable material is arranged to enhance a signal quality of measurements by the one or more sensors through an application of pressure on the digit of the user within an area of the digit adjacent to the one or more sensors.

In some examples of the apparatus described herein, the apparatus may include a hard material extending along one or more portions of the inner circumferential surface of the housing, wherein the deformable material may be disposed on a surface of the hard material, disposed adjacent to the one or more portions of the hard material, or both.

In some examples of the apparatus described herein, an elasticity of the deformable material changes with skin or body temperature to change a shape, a thickness, or both of the deformable material when the wearable ring device may be worn by the user.

In some examples of the apparatus described herein, the deformable material may be elastically deformable such that the deformable material contracts to form the interference fit around the digit of the user when the wearable ring device may be worn by the user and the deformable material expands when the wearable ring device may be off of the digit of the user.

In some examples of the apparatus described herein, the deformable material may be configured to form around the outer circumferential surface of the digit of the user to eliminate air gaps between the outer circumferential surface of the digit of the user and the inner circumferential surface of the housing.

In some examples of the apparatus described herein, the deformable material may be disposed along the one or more portions of the inner circumferential surface of the housing opposite of the one or more sensors.

In some examples of the apparatus described herein, the deformable material may be disposed along the one or more portions of the inner circumferential surface of the housing adjacent to the one or more sensors.

In some examples of the apparatus described herein, the deformable material extends three hundred and sixty degrees around the inner circumferential surface relative to an axis of the housing and the deformable material extends over the one or more sensors.

In some examples of the apparatus described herein, the deformable material comprises optical properties that propagate light from the one or more sensors into the digit of the user.

In some examples of the apparatus described herein, the deformable material may be configured to interface with the housing such that one or more antenna elements disposed within the wearable ring device wirelessly couple one or more components of the wearable ring device with a user device.

In some examples of the apparatus described herein, the deformable material may be disposed over the one or more sensors and the one or more sensors may be configured to perform measurements through the deformable material.

In some examples of the apparatus described herein, the deformable material may be arranged to avoid interfering with measurements by the one or more sensors.

In some examples of the apparatus described herein, the deformable material comprises polyurethane, silicon, a flexible material, a chemical-resistant material, a memory shape polymer material, a soft polymer material, a thermally resistant material, or a combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a housing comprising one or more sensors configured to acquire physiological data from a user; and
a deformable material extending along one or more portions of an inner circumferential surface of the housing and configured to exert pressure on a digit of the user by conforming to an outer circumferential surface of the digit to form an interference fit with the digit of the user when the wearable ring device is worn by the user, wherein the deformable material is arranged to enhance a signal quality of measurements by the one or more sensors through an application of pressure on the digit of the user within an area of the digit adjacent to the one or more sensors.

2. The wearable ring device of claim 1, further comprising:
a hard material extending along one or more portions of the inner circumferential surface of the housing, wherein the deformable material is disposed on a surface of the hard material, disposed adjacent to the one or more portions of the hard material, or both.

3. The wearable ring device of claim 1, wherein an elasticity of the deformable material changes with skin or body temperature to change a shape, a thickness, or both of the deformable material when the wearable ring device is worn by the user.

4. The wearable ring device of claim 1, wherein the deformable material is elastically deformable such that the deformable material contracts to form the interference fit around the digit of the user when the wearable ring device is worn by the user and the deformable material expands when the wearable ring device is off of the digit of the user.

5. The wearable ring device of claim 1, wherein the deformable material is configured to form around the outer circumferential surface of the digit of the user to eliminate air gaps between the outer circumferential surface of the digit of the user and the inner circumferential surface of the housing.

6. The wearable ring device of claim 1, wherein the deformable material is disposed along the one or more portions of the inner circumferential surface of the housing opposite of the one or more sensors.

7. The wearable ring device of claim 1, wherein the deformable material is disposed along the one or more portions of the inner circumferential surface of the housing adjacent to the one or more sensors.

8. The wearable ring device of claim 1, wherein
the deformable material extends three hundred and sixty degrees around the inner circumferential surface relative to an axis of the housing, and
the deformable material extends over the one or more sensors.

9. The wearable ring device of claim 1, wherein the deformable material comprises optical properties that propagate light from the one or more sensors into the digit of the user.

10. The wearable ring device of claim 1, wherein the deformable material is configured to interface with the housing such that one or more antenna elements disposed within the wearable ring device wirelessly couple one or more components of the wearable ring device with a user device.

11. The wearable ring device of claim 1, wherein
the deformable material is disposed over the one or more sensors, and
the one or more sensors are configured to perform measurements through the deformable material.

12. The wearable ring device of claim 1, wherein the deformable material is arranged to avoid interfering with measurements by the one or more sensors.

13. The wearable ring device of claim 1, wherein the deformable material comprises polyurethane, silicon, a flexible material, a chemical-resistant material, a memory shape polymer material, a soft polymer material, a thermally resistant material, or a combination thereof.
